(19) United States
(12) Patent Application Publication
(10) Pub. No.: US 2012/0083719 A1
(43) Pub. Date: Apr. 5, 2012

(54) ULTRASOUND-INTERSECTING BEAMS FOR DEEP-BRAIN NEUROMODULATION

(76) Inventor: David J. Mishelevich, Playa del Rey, CA (US)

(21) Appl. No.: 13/252,054

(22) Filed: Oct. 3, 2011

Related U.S. Application Data
(60) Provisional application No. 61/389,280, filed on Oct. 4, 2010.

(51) Int. Cl. A61N 7/00

(52) U.S. Cl. 601/2

(57) ABSTRACT

Disclosed are methods and devices for ultrasound-mediated non-invasive deep brain neuromodulation impacting one or a plurality of points in a neural circuit using intersecting ultrasound beams. Depending on the application, this can produce short-term effects (as in the treatment of post-surgical pain) or long-term effects in terms of Long-Term Potentiation (LTP) or Long-Term Depression (LTD) to treat indications such as neurologic and psychiatric conditions. Multiple beams intersect and summate at one or a plurality of targets. The ultrasound transducers are used with control of direction of the energy emission, intensity, frequency (carrier frequency and/or neuromodulation frequency), pulse duration, pulse pattern, and phase/intensity relationships to targeting and accomplishing up-regulation and/or down-regulation.

### CROSS REFERENCE TO RELATED APPLICATIONS

This non-provisional patent application claims priority to provisional patent application 61/389,280 of the same name filed on 2010 Oct. 4.

### INCORPORATION BY REFERENCE

All publications, including patents and patent applications, mentioned in this specification are herein incorporated by reference in their entirety to the same extent as if each individual publication was specifically and individually cited to be incorporated by reference.

### FIELD OF THE INVENTION

Described herein are systems and methods for Ultrasound Stimulation including one or a plurality of ultrasound sources for stimulation of target deep brain regions to up-regulate or down-regulate neural activity.

### BACKGROUND OF THE INVENTION

It has been demonstrated that focused ultrasound directed at neural structures can stimulate those structures. If neural activity is increased or excited, the neural structure is said to be up regulated; if neural activated is decreased or inhibited, the neural structure is said to be down regulated. Down regulation means that the firing rate of the neural target has its firing rate decreased and thus is inhibited and up regulation means that the firing rate of the neural target has its firing rate increased and thus is excited. Neural structures are usually assembled in circuits. For example, nuclei and tracts connecting them make up a circuit. The potential application of ultrasonic therapy of deep-brain structures has been suggested previously (Gavrilov L R, Tsirulnikov E M, and I A Davies, “Application of focused ultrasound for the stimulation of neural structures,” Ultrasound Med Biol. 1996; 22(2):179-92. and S. J. Norton, “Can ultrasound be used to stimulate nerve tissue?,” BioMedical Engineering OnLine 2003, 2:6). Norton notes that while Transcranial Magnetic Stimulation (TMS) can be applied within the head with greater intensity, the gradients developed with ultrasound are comparable to those with TMS. It was also noted that monophasic ultrasound pulses are more effective than biphasic ones. Instead of using ultrasonic stimulation alone, Norton applied a strong DC magnetic field as well and describes the mechanism as that given that the tissue to be stimulated is conductive that particle motion induced by an ultrasonic wave will induce an electric current density generated by Lorentz forces.

The effect of ultrasound is at least two fold. First, increasing temperature will increase neural activity. An increase up to 42 degrees C. (say in the range of 39 to 42 degrees C.) locally for short time periods will increase neural activity in a way that one can do so repeatedly and be safe. One needs to make sure that the temperature does not rise about 50 degrees C. or tissue will be destroyed (e.g., 56 degrees C. for one second). This is the objective of another use of therapeutic application of ultrasound, ablation, to permanently destroy tissue (e.g., for the treatment of cancer). An example is the ExAblate device from InSightec in Haifa, Israel. The second mechanism is mechanical perturbation. An explanation for this has been provided by Tyler et al. from Arizona State University (Tyler, W. J., Y. Tufail, M. Finsterwald, M. L. Tauchmann, E. J. Olsen, C. Majestic, “Remote excitation of neuronal circuits using low-intensity, low-frequency ultrasound,” PLoS One 3(10): e3511, doi:10.137/1/journal.pone.0003511, 2008)) where voltage gating of sodium channels in neural membranes was demonstrated. Pulsed ultrasound was found to cause mechanical opening of the sodium channels that resulted in the generation of action potentials. Their stimulation is described at Low Intensity Low Frequency Ultrasound (LILFU). They used bursts of ultrasound at frequencies between 0.44 and 0.67 MHz, lower than the frequencies used in imaging. Their device delivered 23 milliwatts per square centimeter of brain—a fraction of the roughly 180 mW/cm² upper limit established by the U.S. Food and Drug Administration (FDA) for womb-scanning sonograms; thus such devices should be safe to use on patients. Ultrasound impact to open calcium channels has also been suggested. The above approach is incorporated in a patent application submitted by Tyler (Tyler, Tyler, William, James P., PCT/US2009/050560, WO 2010/009141, published 2011 Jan. 21).

Alternative mechanisms for the effects of ultrasound may be discovered as well. In fact, multiple mechanisms may come into play, but, in any case, this would not effect this invention.

Approaches to date of delivering focused ultrasound vary. Bystritsky (U.S. Pat. No. 7,283,861, Oct. 16, 2007) provides for focused ultrasound pulses (FUP) produced by multiple ultrasound transducers (said preferably to number in the range of 300 to 1000) arranged in a cap place over the skull to affect a multi-beam output. These transducers are coordinated by a computer and used in conjunction with an imaging system, preferable an fMRI (functional Magnetic Resonance Imaging), but possibly a PET (Positron Emission Tomography) or V-EEG (Video-Electroencephalography) device. The user interacts with the computer to direct the FUP to the desired point in the brain, sees where the stimulation actually occurred by viewing the imaging result, and thus adjusts the position of the FUP according. The position of focus is obtained by adjusting the phases and amplitudes of the ultrasound transducers (Clement and Hynynen, “A non-invasive method for focusing ultrasound through the human skull,” Phys. Med. Biol. 47 (2002) 1219-1236). The imaging also illustrates the functional connectivity of the target and surrounding neural structures. The focus is described as two or more centimeters deep and 0.5 to 1000 mm in diameter or preferably in the range of 2-12 cm deep and 0.5-2 mm in diameter. Either a single FUP or multiple FUPs are described as being able to be applied to either one or multiple live neuronal circuits. It is noted that differences in FUP phase, frequency, and amplitude produce different neural effects. Low frequencies (defined as below 500 Hz.) are inhibitory. High frequencies (defined as being in the range of 500 Hz to 5 MHz) are excitatory and activate neural circuits. This works whether the target is gray or white matter. Repeated sessions result in long-term effects. The cap and transducers to be employed are preferably made of non-ferrous material to reduce image distortion in fMRI imaging. It was noted that if after treatment the reactivity as judged with fMRI of the patient with a given condition becomes more like that of a normal patient, this may be indicative of treatment effectiveness. The FUP is to be applied 1 ms to 1 s before or after the imaging. In addition a CT (Computed Tomography) scan can be run to gauge the bone density and structure of the skull.

Deisseroth and Schneider describe an alternative approach (U.S. patent application Ser. No. 12/263,026 published as US 2009/0112133 A1, Apr. 30, 2009). Neural transmission patterns are modified between neural structures and/or regions using sound (including use of a curved transducer and a lens) or RF. The impact of Long-Term Potentiation (LTP) and Long-Term Depression (LTD) for durable effects is emphasized. It is noted that sound produces stimulation by both thermal and mechanical impacts. The use of ionizing radiation also appears in the claims.

Adequate penetration of ultrasound through the skull has been demonstrated (Hynynen, K. and F A Jolesz, “Demonstration of potential noninvasive ultrasound brain therapy through an intact skull,” Ultrasound Med Biol, 1998 February; 24(2):275-83 and Clement G T, Hynynen K (2002) A non-invasive method for focusing ultrasound through the human skull. Phys Med Biol 47: 1219-1236.). Ultrasound can be focused to 0.5 to 2 mm as TMS to 1 cm at best.

The targeting can be done with one or more of known external landmarks, an atlas-based approach (e.g., Tailarach or other atlas used in neurosurgery) or imaging (e.g., fMRI or Positron Emission Tomography). The imaging can be done as a one-time set-up or at each session although not using imaging or using it sparingly is a benefit, both functionally and the cost of administering the therapy, over Bystritsky (U.S. Pat. No. 7,283,861) which teaches consistent concurrent imaging.

While ultrasound can be focused down to a diameter on the order of one to a few millimeters (depending on the frequency), whether such a tight focus is required depends on the conformation of the neural target. For example, some targets, like the Cingulate Gyrus, are elongated and will be more effectively served with an elongated ultrasound field at the target.

### SUMMARY OF THE INVENTION

It is the purpose of this invention to provide an ultrasound device delivering enhanced non-invasive deep brain or superficial deep-brain neuromodulation impacting one or a plurality of points in a neural circuit to produce acute effects or Long-Term Potentiation (LTP) or Long-Term Depression (LTD) using up-regulation or down-regulation.

### BRIEF DESCRIPTION OF THE DRAWINGS

FIG. 1: Flat ultrasound transducer producing a parallel beam.

FIG. 2: Three flat ultrasound transducers using global ultrasound conduction medium with beams intersecting on a Dorsal Anterior Cingulate Gyms (DACG) target.

FIG. 3: Three flat ultrasound transducers using individual ultrasound conduction media with beams intersecting on a Dorsal Anterior Cingulate Gyms (DACG) target.

FIG. 4: Two sets of flat ultrasound transducers using global ultrasound conduction medium with beams intersecting on Dorsal Anterior Cingulate Gyms (DACG) and Insula targets.

FIG. 5: Block diagram of the mechanism for controlling the multiple ultrasound beams.

### DETAILED DESCRIPTION OF THE INVENTION

The invention is an ultrasound device using intersecting beams delivering enhanced non-invasive deep brain or superficial deep-brain neuromodulation impacting one or a plurality of points in a neural circuit to produce acute effects (as in the treatment of post-surgical pain) or Long-Term Potentiation (LTP) or Long-Term Depression (LTD) using up-regulation or down-regulation.

The stimulation frequency for inhibition as below 500 Hz (depending on condition and patient). The stimulation frequency for excitation is in the range of 500 Hz to 5 MHz. There is not a sharp border at 500 Hz, however. In this invention, the ultrasound acoustic frequency is in range of 0.3 MHz to 0.8 MHz to permit effective transmission through the skull with power generally applied less than 180 mW/cm² but also at higher target- or patient-specific levels at which no tissue damage is caused. The acoustic frequency (e.g., 0.44 MHz that permits the ultrasound to effectively penetrate through skull and into the brain) is gated at the lower rate to impact the neuronal structures as desired (e.g., say 300 Hz for inhibition (down-regulation) or 1 kHz for excitation (up-regulation). The modulation frequency (superimposed on the carrier frequency of say 0.5 MHz or similar) may be divided into pulses 0.1 to 20 msec. repeated at frequencies of 2 Hz or lower for down regulation and higher than 2 Hz for up regulation) although this will be both patient and condition specific. If there is a reciprocal relationship between two neural structures (i.e., if the firing rate of one goes up the firing rate of the other will decrease), it is possible that it would be appropriate to hit the target that is easiest to obtain the desired result. For example, one of the targets may have critical structures close to it so if it is a target that would be down regulated to achieve the desired effect, it may be preferable to up-regulate its reciprocal more-easily-accessed or safer reciprocal target instead. The frequency range allows penetration through the skull balanced with good neural-tissue absorption. Ultrasound therapy can be combined with therapy using other devices (e.g., Transcranial Magnetic Stimulation (TMS), transcranial Direct Current Stimulation (tDCS), and/or Deep Brain Stimulation (DBS) using implanted electrodes, optogenetics, radiosurgery, Radio-Frequency (RF)), behavioral therapy, or medications.

The lower bound of the size of the spot at the point of focus will depend on the ultrasonic frequency, the higher the frequency, the smaller the spot. Ultrasound-based neuromodulation operates preferentially at low frequencies relative to say imaging applications so there is less resolution. As an example, let us have a hemispheric transducer with a diameter of 3.8 cm. At a depth approximately 7 cm the size of the focused spot will be approximately 4 mm at 500 kHz where at 1 Mhz, the value would be 2 mm. Thus in the range of 0.4 MHz to 0.7 MHz, for this transducer, the spot sizes will be on the order of 5 mm at the low frequency and 2.8 mm at the high frequency.

Transducer array assemblies of the type used in this invention may be supplied to custom specifications by Imasonic in France (e.g., large 2D High Intensity Focused Ultrasound (HIFU) hemispheric array transducer)(Fleury G., Berriet, R., Le Baron, O., and B. Huguenin, “New piezocomposite transducers for therapeutic ultrasound,” 2^{nd} International Symposium on Therapeutic Ultrasound-Seattle-31/07-Feb. 8, 2002), typically with numbers of sound transducers of 300 or more. Blatek and Keramos-Etalon in the U.S. are other custom-transducer suppliers. The power applied will determine whether the ultrasound is high intensity or low intensity (or medium intensity) and because the sound transducers are custom, any mechanical or electrical changes can be made, if and as required.

The locations and orientations of the transducers in this invention can be calculated by locating the applicable targets relative to atlases of brain structure such as the Tailarach atlas or established though fMRI, PET, or other imaging of the head of a specific patient. Using multiple ultrasound transducers two or more targets can be targeted simultaneously or sequentially. The ultrasonic firing patterns can be tailored to the response type of a target or the various targets hit within a given neural circuit.

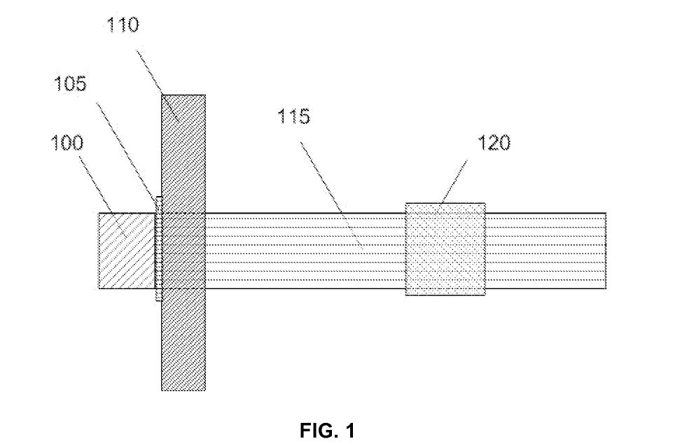

FIG. 1 shows a flat ultrasound transducer producing a parallel beam intersecting a single target. Flat ultrasound transducer 100 produces ultrasound beam 115. To be practical, ultrasound beam 115 passes through skull section 110 with coupling medium 105 interposed between transducer 100 and skull section 110 to support effective transmission. Ultrasound beam 115 hits target 120.

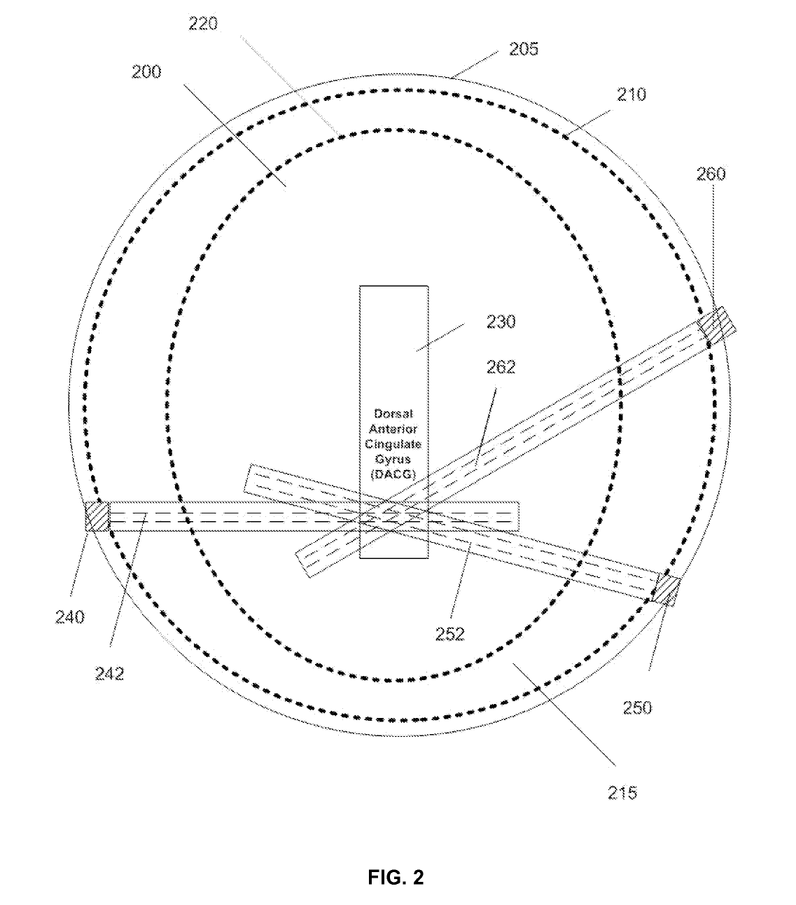

FIG. 2 illustrates head 200 containing target Dorsal Anterior Cingulate Gyms (DACG) 230. Frame 205 holds three ultrasound transducers 240, 250, 260. The beam from each ultrasound transducer passes though an ultrasound-conduction medium 215 with ultrasound-conduction gel interfaces 210 at the transducer face and 220 at the head. Ultrasound transducer 240 generates ultrasound beam 242, ultrasound transducer 250 generates ultrasound beam 252, and ultrasound transducer 260 generates ultrasound beam 262. Ultrasound beams 242, 252, and 262 intersect at Dorsal Anterior Cingulate Gyms target 230 and neuromodulate the DACG. The effects of beams 242, 252, and 262 are additive. Examples of ultrasound conduction media include Dermasol from California Medical Innovations and silicone oil in a containment pouch. Ultrasound-conjunction gel (not shown) can be placed just at the interfaces between any of the ultrasound transducers and the band of ultrasonic-conduction medium 215 and that band and head 200 as long as the beam regions are covered. One or more of the plurality of the ultrasound transducers can also be used with an acoustic lens (not shown). For elongated targets such as the DACG, the intersecting beams can be spread to cover a broader neural region. In addition the width of the ultrasound transducer and thus the width of the beam can be varied.

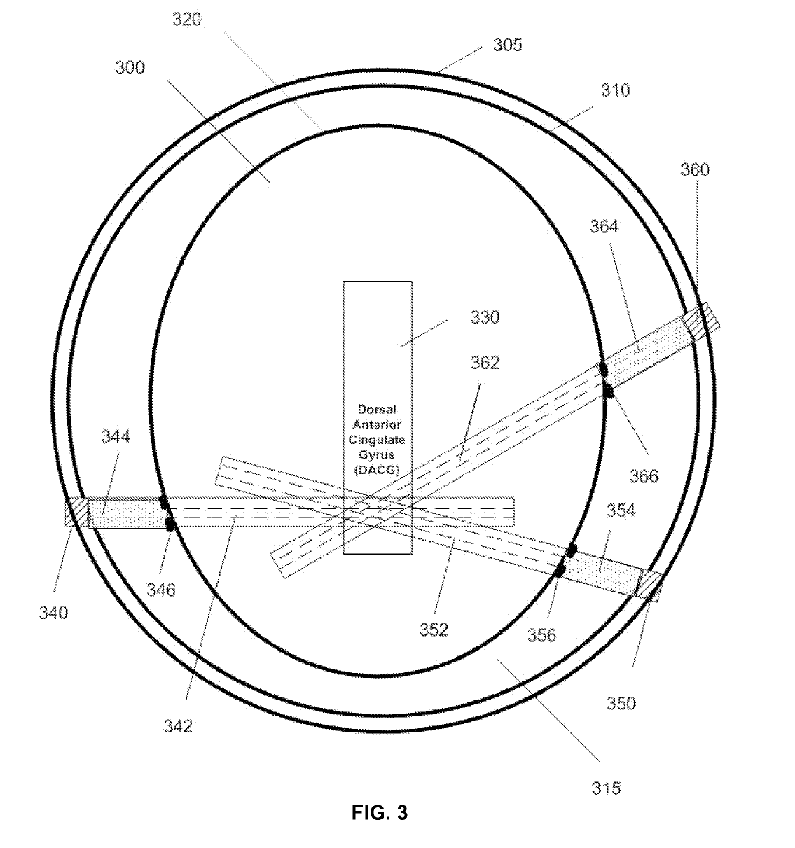

In another embodiment, the ultrasound-conduction medium is not incorporated in a continuous band around the head (215 in FIG. 2), but instead is configured as a single ultrasound conduction medium for each ultrasound transducer. FIG. 3 illustrates head 300 containing target Dorsal Anterior Cingulate Gyms (DACG) 330. Frame 305 holds three ultrasound transducers 340, 350, 360. The beam from each ultrasound transducer passes though individual ultrasound-conduction media. For ultrasound transducer 340, beam 342 passes through ultrasound-conduction medium 344 and then through ultrasound-conduction gel 346 at the interface with head 300. There also can be a layer ultrasound-conduction gel (not shown) at the interface between ultrasound transducer 340 and ultrasound-conduction medium 344. For ultrasound transducer 350, beam 352 passes through ultrasound-conduction medium 354 and then through ultrasound-conduction gel 356 at the interface with head 300. There also can be a layer of ultrasound-conduction gel (not shown) at the interface between ultrasound transducer 350 and ultrasound-conduction medium 354. In like manner, for ultrasound transducer 360, beam 362 passes through ultrasound-conduction medium 364 and then through ultrasound-conduction gel 366 at the interface with head 300. There also can be a layer of ultrasound-conduction gel (not shown) at the interface between ultrasound transducer 360 and ultrasound-conduction medium 364. Ultrasound beams 342, 352, and 362 intersect at Dorsal Anterior Cingulate Gyms target 330 and neuromodulate the DACG. The effects of beams 342, 342, and 362 are additive. Each ultrasound transducer can also be used with an acoustic lens (not shown). For elongated targets such as the DACG, the intersecting beams can be spread to cover a broader neural region. In addition the width of the ultrasound transducer and thus the width of the beam can be varied.

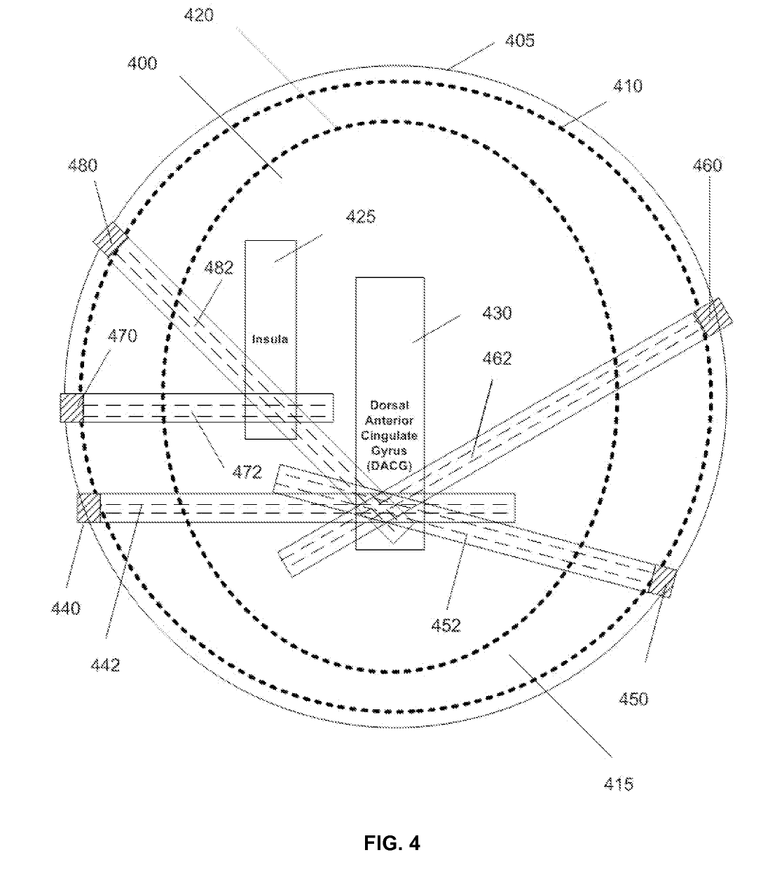

In another embodiment, a plurality of targets is each hit by intersecting ultrasound beams. FIG. 4 illustrates head 400 containing targets Insula 425 and Dorsal Anterior Cingulate Gyms (DACG) 430. Frame 405 holds five ultrasound transducers 440, 450, 460, 470, 480. The beam from each ultrasound transducer passes though a band of ultrasound-conduction medium 415 although in an alternative embodiment the beams can pass through individual ultrasound-conduction media such as shown in FIG. 3. From ultrasound transducer 440, beam 442 passes through ultrasound-conduction medium 415 then into the head, hitting target DACG 430. From ultrasound transducer 450, beam 452 passes through ultrasound-conduction medium 415 then into the head, hitting target DACG 430. In like manner, from ultrasound transducer 460, beam 462 passes through ultrasound-conduction medium 415 then into the head, hitting target DACG 430. Beams 442, 452, and 462 intersect in the Dorsal Anterior Cingulate Gyms 430, enhancing the neuromodulation at that target. Effects of beams 442, 452, and 462 are additive. Ultrasound-conjunction conjunction gel (not shown) can be placed just at the interfaces between any of the ultrasound transducers and the band of ultrasonic-conduction medium 415 and that band and head 400 as long as the beam regions are covered. The other neural target in FIG. 4 is the Insula 425. Targeting the Insula are ultrasound transducers 470 and 480. From ultrasound transducer 470, beam 472 passes through ultrasound-conduction medium 415 then into the head, hitting target Insula 425. From ultrasound transducer 480, beam 482 passes through ultrasound-conduction medium 415 then into the head, hitting target Insula 425. It also will intersect Dorsal Anterior Cingulate Gyms 430 but will have minimal impact because it will be the only ultrasound beam present where it passes through the DACG. Beams 472 and 482 intersect in the Insula 425, enhancing the neuromodulation at that target. Beams 472 and 482 are additive. Beam 482 not only neuromodulates the target Insula 425, but also continues through to neuromodulate DACG 430 where beam 482 intersects beams 442, 452, and 462 from ultrasound transducers 440, 450, and 460. The effects of beams 442, 452, 462, and 482 are additive. The ultrasound transducers can also be used with an acoustic lens (not shown). Again, for elongated targets such as the DACG, the intersecting beams can be spread to cover a broader neural region. In addition the width of the ultrasound transducer and thus the width of the beam can be varied.

In another embodiment, the neuromodulation of one or a plurality of ultrasound transducers is combined with the neuromodulation from one or a plurality of Transcranial Magnetic Stimulation (TMS) electromagnetic coils. In another embodiment, a viewing hole can be placed in an ultrasound transducer to provide an imaging port. Blatek, Imasonic and Keramos-Etalon can supply such configurations.

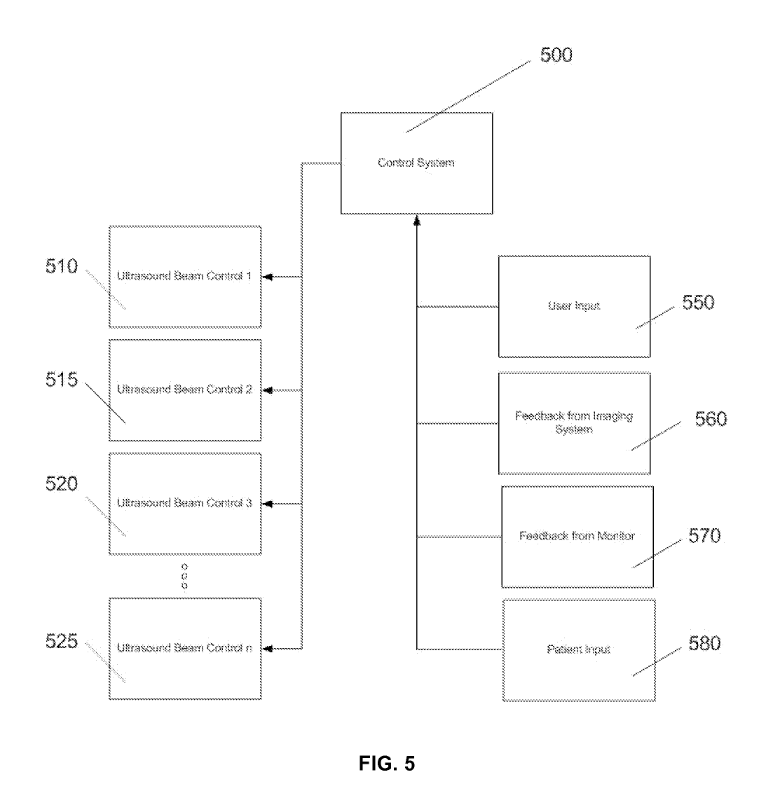

FIG. 5 shows a control block diagram. The direction of the energy emission, intensity, frequency (carrier frequency and/or neuromodulation frequency), pulse duration, pulse pattern, and phase/intensity relationships in targeting for the ultrasonic transducers 510, 515, 520, 525 (and, as applicable, additional ultrasound transducers as indicated by the ellipsis between ultrasound transducers 520 and 525) are controlled by control system 500 with control input from user by user input 550 and/or from feedback from imaging system 560 (either automatically or display to the user with actual control through user input 550), and/or feedback from a monitor (sound and/or thermal) 570, and/or the patient 580 and/or, in the future, other feedback. If positioning of the ultrasound transducers is included as a control element, then control system 550 will control positioning as well.

The invention can be applied to a number of conditions including, but not limited to, addiction, Alzheimer's Disease, anorgasmia, anhedonia, Attention Deficit Hyperactivity Disorder, Autism Spectrum Disorders, Huntington's Chorea, Impulse Control Disorder, OCD, Social Anxiety Disorder, Parkinson's Disease and other motor disorders, Post-Traumatic Stress Disorder, depression, bipolar disorder, pain, insomnia, spinal cord injuries, gastrointestinal motility disorders, neuromuscular disorders, tinnitus, panic disorder, Tourette's Syndrome, amelioration of brain cancers, dystonia, obesity, stuttering, ticks, head trauma, stroke, and epilepsy. In addition it can be applied to cognitive enhancement, hedonic stimulation, enhancement of neural plasticity, improvement in wakefulness, brain mapping, diagnostic applications, and other research functions. In addition to stimulation or depression of individual targets, the invention can be used to globally depress neural activity that can have benefits, for example, in the early treatment of head trauma or other insults to the brain.

All of the embodiments above, except those explicitly restricted in configuration to hit a single target, are capable of and usually would be used for targeting multiple targets either simultaneously or sequentially. Hitting multiple targets in a neural circuit in a treatment session is an important component of fostering a durable effect through Long-Term Potentiation (LTP) and/or Long-Term Depression (LTD) or enhances acute effects (e.g., such as treatment of post-surgical pain). In addition, this approach can decrease the number of treatment sessions required for a demonstrated effect and to sustain a long-term effect. Follow-up tune-up sessions at one or more later times may be required. In some cases, the neural structures will be targeted bilaterally (e.g., both the right and the left Insula) and in others only one side will targeted (e.g., the right Insula in the case of addiction).

The invention allows stimulation adjustments in variables such as, but not limited to, intensity, firing pattern, and frequency, and position to be adjusted so that if a target is in two neuronal circuits the output of the transducer or transducers can be adjusted to get the desired effect and avoid side effects. Position can be adjusted as well. The side effects could occur because for one indication the given target should be up regulated and for the other down regulated. An example is where a target or a nearby target would be down regulated for one indication such as pain, but up-regulated for another indication such as depression. This scenario applies to either the Dorsal Anterior Cingulate Gyms (DACG) or Caudate Nucleus. Even when a common target is neuromodulated, adjustment of stimulation parameters may moderate or eliminate a problem.

The invention also covers contradictory effects in cases where a target is common to both two neural circuits in another way. This is accomplished by treating (either simultaneously or sequentially, as applicable) other neural-structure targets in the neural circuits in which the given target is a member to counterbalance contradictory side effects. This also applies to situations where a tissue volume of neuromodulation encompasses a plurality of targets. Again, an example is where a target or a nearby target would be down regulated for one indication such as pain, but up-regulated for another indication such as depression. This scenario applies to the Dorsal Anterior Cingulate Gyms (DACG). To counterbalance the down regulation of the DACG during treatment for pain that negatively impacts the treatment for depression, one would up regulate the Nucleus Accumbens or Hippocampus that are other targets in the depression neural circuit. A plurality of such applicable targets could be stimulated as well.

Another applicable scenario is the Nucleus Accumbens that is down regulated to treat addiction, but up regulated to treat depression. To counteract the down regulation of the Nucleus Accumbens to treat depression but will negatively impact the treatment of depression that would like the Nucleus Accumbens to be up regulated, one would up regulate the Caudate Nucleus as well. Not only can potential positive impacts be negated, one wants to avoid side effects such as treating depression, but also causing pain. These principles of the invention are applicable whether ultrasound is used alone, in combination with other modalities, or with one or more other modalities of treatment without ultrasound. Any modality involved in a given treatment can have its stimulation characteristics adjusted in concert with the other involved modalities to avoid side effects.

The various embodiments described above are provided by way of illustration only and should not be construed to limit the invention. Based on the above discussion and illustrations, those skilled in the art will readily recognize that various modifications and changes may be made to the present invention without strictly following the exemplary embodiments and applications illustrated and described herein. Such modifications and changes do not depart from the true spirit and scope of the present invention.

What is claimed is:

1. A method for ultrasound neuromodulation of one or a plurality of deep-brain targets comprising:
a. attaching a plurality of ultrasound transducers to a positioning frame, and
b. aiming the beams from the ultrasound transducers so said beams intersect at the one or plurality of targets,
whereby the combination of said ultrasound beams neuromodulates the targeted neural structures producing one or a plurality of regulations selected from the group consisting of up-regulation and down-regulation.

2. The method of claim 1, wherein the width of the ultrasound transducer and resultant beam are matched to the size of the target.

3. The method of claim 1, wherein a plurality of ultrasound transducers is employed to neuromodulate multiple targets in multiple neural circuits.

4. The method of claim 1, wherein one or a plurality of ultrasound transducers is used with control of selected from the group consisting of direction of the energy emission, intensity, frequency (carrier frequency and/or neuromodulation frequency), pulse duration, pulse pattern, and phase/intensity relationships to targeting.

5. The method of claim 1, wherein one or plurality of targets is up regulated and one or a plurality of targets is down regulated.

6. The method of claim 1, wherein one or a plurality of targets is hit with a single ultrasound beam.

7. The method of claim 1, wherein a combination of a plurality of ultrasound transducers and Transcranial Magnetic Stimulation electromagnets is employed to neuromodulate one or a plurality of targets in one or a plurality of neural circuits.

8. The method of claim 1 wherein ultrasound therapy is combined with or replaced by one of more therapies selected from the group consisting of Transcranial Magnetic Stimulation (TMS), transcranial Direct Current Stimulation (tDCS), Deep-Brain Stimulation (DBS) using implanted electrodes, application of optogenetics, radiosurgery, Radio-Frequency (RF) therapy, behavioral therapy, and medications.

9. The method of claim 1, wherein the effect is selected from one or more of the group consisting of acute effect, Long-Term Potentiation, Long-Term Depression.

10. A device for ultrasound neuromodulation of one or a plurality of deep-brain targets comprising:
a. attaching a plurality of ultrasound transducers to a positioning frame, and
b. aiming the beams from the ultrasound transducers so said beams intersect at the one or plurality of targets,
whereby the combination of said ultrasound beams neuromodulates the targeted neural structures producing one or a plurality of regulations selected from the group consisting of up-regulation and down-regulation.

11. The device of claim 10, wherein the width of the ultrasound transducer and resultant beam are matched to the size of the target.

12. The device of claim 10, wherein a plurality of ultrasound transducers is employed to neuromodulate multiple targets in multiple neural circuits.

13. The device of claim 10, wherein one or a plurality of ultrasound transducers is used with control of selected from the group consisting of direction of the energy emission, intensity, frequency (carrier frequency and/or neuromodulation frequency), pulse duration, pulse pattern, and phase/intensity relationships to targeting.

14. The device of claim 10, wherein one or plurality of targets is up regulated and one or a plurality of targets is down regulated.

15. The device of claim 10, wherein a plurality of targets is hit with a single ultrasound beam.

16. The device of claim 10, wherein a combination of a plurality of combination ultrasound transducer and Transcranial Magnetic Stimulation electromagnets is employed to neuromodulate one or a plurality of targets in one or a plurality of neural circuits.

17. The device of claim 10, wherein ultrasound therapy is combined with or replaced by one of more therapies selected from the group consisting of Transcranial Magnetic Stimulation (TMS), transcranial Direct Current Stimulation (tDCS), Deep-Brain Stimulation (DBS) using implanted electrodes, application of optogenetics, radiosurgery, Radio-Frequency (RF) therapy, behavioral therapy, and medications.

18. The device of claim 10, wherein the effect is selected from one or more of the group consisting of acute effect, Long-Term Potentiation, Long-Term Depression.
